(19) United States
(12) Patent Application Publication
(10) Pub. No.: US 2008/0160556 A1
(43) Pub. Date: Jul. 3, 2008

(54) Bioassay element and producing method thereof

(75) Inventor: Reiko Ohara, Hsinchu (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 12/073,832

(22) Filed: Mar. 11, 2008

Related U.S. Application Data
(63) Continuation-in-part of application No. 11/637,825, filed on Dec. 13, 2006.

(30) Foreign Application Priority Data
Dec. 18, 2007 (TW) 96148382

(51) Int. Cl. C12Q 1/46; C12M 1/00; C12Q 1/00

(52) U.S. Cl. 435/20; 435/287.9; 435/4

(57) ABSTRACT

The present invention relates to a low-priced bioassay element, comprising: a carrier comprising a porous cellulose filter paper and a stationary layer, wherein said stationary layer comprises casein and calcium ions, and covers on said porous cellulose filter paper; a coating film comprising casein and calcium ions, which covers on the surface of said carrier; and an enzyme dispersed in said coating film; in which the activity of the enzyme comprised in the bioassay element can be maintained for a long time, and the assay result can be read by naked eyes or a spectrophotometer.

### CROSS REFERENCES TO THE RELATED APPLICATIONS

This is a Continuation-in-part of U.S. application Ser. No. 11/637,825, filed Dec. 13, 2006, now pending.

### BACKGROUND OF THE INVENTION

1. Field of the Invention

The present invention relates to a low-priced bioassay element and the producing method thereof, in which the activity of the enzyme comprised in the bioassay element can be maintained for a long time, and the assay result can be read by naked eyes or a spectrophotometer.

2. Description of the Related Art

In order to solve a problem associated with pesticide residues in commercial vegetables and fruits, it is necessary to develop a method to screen pesticide residues rapidly and thus to invent a disposable, cheap bioassay kit for detecting pesticide residues immediately.

In U.S. Pat. No. 4,324,858, the reaction of an enzyme and a mixed solution of indoxyl acetate, potassium ferricyanide and potassium ferrocyanide trihydrate is used to detect the existence of pesticides, and the substrate material disclosed in this invention is ion exchange paper. And, the enzyme reaction mechanism used in another patent, U.S. Pat. No. 3,049,411, is the same as that in U.S. Pat. No. 4,324,858, but the substrate material is organic glass fiber paper.

In addition, enzyme immobilization is a critical technique for the development of a bioassay kit. Compared with enzymes in a “solution” state, the decrease of enzyme activity of enzymes immobilized in a “dry” state is slower. So the enzyme immobilization is highly advantageous for extending the preservative time of the bioassay kit. In the prior art, however, the activity of the enzyme immobilized and adsorbed on the carrier surface is approximately one tenth of the total activity of the original enzyme. In other words, enzyme immobilization dramatically declines the enzyme activity. As the detection of a bioassay kit is based on a calorimetric process, such as Ellman test, the enzyme and/or the calorimetric products may be eluted out when the enzyme-immobilized carrier is dipped in an aqueous substrate solution, thereby resulting in reduction of chromaticity sensitivity. There are a variety of methods of enzyme immobilization, including physical adsorption, ion binding, covalent binding, cross-linking, entrapments, etc. According to the method of enzyme immobilization in U.S. Pat. No. 5,624,831, gelatin and trehalose are used to form a stable film through the synergistic effect of these two materials, and the enzyme can be successfully entrapped in the film and preserved up to 31 days under a dry condition at 4° C., room temperature, or even 50° C., in which the enzyme activity is maintain at 100%. Nevertheless, the activity of the enzyme immobilized by this method may reduce to 70% after preserving 31 days under a dry condition at 37° C.

Due to the relatively large consumption of disposable pesticide residues test strips, those high-priced substrate materials used in the prior art have caused a cost burden. Therefore, developing a disposable pesticide residues test strip with advantages of low cost, high sensitivity and convenience for storage and result reading to detect will be beneficial for food safety.

### SUMMARY OF THE INVENTION

To solve the above-mentioned problems, the main object of the present invention is to provide a low-priced bioassay element, in which the activity of the enzyme therein can be maintained for a long time, and the assay result can be read by naked eyes or a spectrophotometer. In comparison with the conventional bioassay elements, the bioassay element of the present invention uses a porous cellulose filter paper as substrate, which costs one fourth to one fifth of the substrates disclosed in the prior art, so the production cost of the present invention is greatly lowered. In addition, the bioassay element provided by the present invention obviously enhances pesticide detection sensitivity, and the color changes can be observed by naked eyes or a spectrophotometer to determine whether pesticides exist. This convenient element can be used for in-time detection in the work field.

Another object of the present invention is to provide a method of producing the bioassay element highly sensitive to pesticide, in which the method is simpler and cheaper than the conventional method.

To achieve these objects, the present invention provides a bioassay element, comprising:
a carrier comprising a porous cellulose filter paper and a stationary layer, wherein said stationary layer comprises casein and calcium ions, and covers on said porous cellulose filter paper;
a coating film comprising casein and calcium ions, which covers on the surface of said carrier; and
an enzyme dispersed in said coating film.

In the preferred embodiments, said porous cellulose filter paper has a pore size of 10 μm to 30 μm; more preferably, 23 μm to 30 μm.

In the preferred embodiments, said enzyme is acetylcholinesterase.

In the preferred embodiments, said coating film further comprises a color developer.

In the preferred embodiments, said coating film further comprises trehalose.

The present invention also provides a method of producing the above-mentioned bioassay element, which comprises steps of:
(a) providing a porous cellulose filter paper;
(b) coating a mixed solution on said porous cellulose filter paper, wherein said mixed solution comprises casein and calcium ions;
(c) drying said mixed solution to form a stationary layer;
(d) coating a bioassay solution on said stationary layer, wherein said bioassay solution comprises an enzyme, casein and calcium ions; and
(e) drying said bioassay solution.

In the preferred embodiments, said enzyme is acetylcholinesterase. In the preferred embodiments, said mixed solution of step (c) is dried by means of air drying, vacuum drying, suction drying, or a combination thereof; more preferably, by suction drying.

In the preferred embodiments, said mixed solution of step (b) is prepared by mixing 2% to 4% of casein solution and 150 mM to 300 mM of a calcium ion solution in a ratio of 14:1 to 11:1 by volume; more preferably, by mixing 4% of casein solution and 300 mM of a calcium ion solution in a ratio of 14:1 by volume.

In the preferred embodiments, said bioassay solution of step (e) is dried by means of air drying, vacuum drying, suction drying, or a combination thereof; more preferably, by the combination of air drying, vacuum drying and suction drying.

In the preferred embodiments, said bioassay solution of step (d) is prepared by mixing a mixed solution of enzyme and a mixed solution of casein in a ratio of 5:7 to 5:8.75 by volume; more preferably, in a ratio of 5:7 by volume.

In the preferred embodiments, said mixed solution of enzyme is prepared by mixing 0.4 U/μL to 1.52 U/μL of an enzyme solution and 5% of trehalose solution in a ratio of 2:108 to 3:108 by volume; more preferably, by mixing 1.52 U/μL of an enzyme solution and 5% of trehalose solution in a ratio of 2.61:108 by volume.

In the preferred embodiments, said mixed solution of casein is prepared by mixing 10 mM of color developer DTNB, 4% to 10% of casein solution and 120 mM to 320 mM of a calcium ion solution in a ratio of 0.009˜0.012:6˜7.75:1 (w/v/v); more preferably, by mixing 10 mM of color developer DTNB, 8% of casein solution and 240 mM of a calcium ion solution in a ratio of 0.009:6:1 (w/v/v).

In summary, the present invention provides a low-priced bioassay element and the producing method thereof, in which the activity of the enzyme therein can be maintained for a long time and the assay result can be read by naked eyes or a spectrophotometer. The bioassay element of the present invention uses a porous cellulose filter paper as the substrate, so the production cost is greatly lowered and the pesticide detection sensitivity is enhanced. Furthermore, the reaction mechanism applied to the present invention results in color changes, which is easy for result reading, and those non-professionals can detect pesticide residues by low-priced bioassay elements. Therefore, the detection of pesticide residues can be performed in time, and effectively stops the transportation of vegetables and fruits with pesticide residues. This is a great advantage for food safety.

### BRIEF DESCRIPTION OF THE DRAWINGS

FIG. 1 is the structure illustration of the bioassay element of the present invention.

### DETAILED DESCRIPTION OF THE PREFERRED EMBODIMENTS

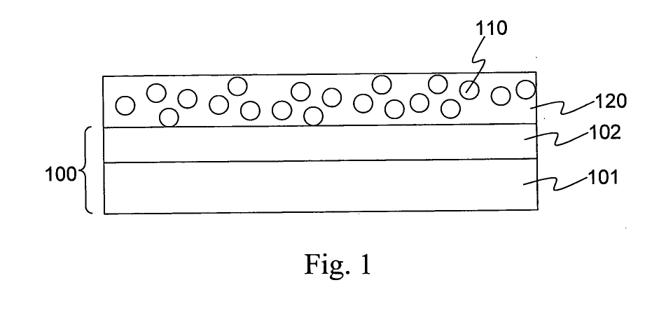

As shown in FIG. 1, the bioassay element 10 provided by the present invention, which is used for detecting chemical residues in samples, comprises a carrier 100, a coating film 120 and an enzyme 110, wherein the carrier 100 comprises a porous cellulose filter paper 101 and a stationary layer 102 and the stationary layer 102 covers on the porous cellulose filter paper 101; the coating film 120 covers on the surface of the stationary layer 102; and the enzyme 110 is dispersed in the coating film 120. Both the coating film 120 and the stationary layer 102 comprise casein and calcium ions, thus the immobilized amount of the enzyme 110 is effectively increased, and the activity of the enzyme 110 can be maintained. Additionally, the coating film 120 can further comprise the color developer DTNB and trehalose, wherein the color developer enables the user to read the assay results by naked eyes, and the trehalose is added in the bioassay element of the present invention as an enzyme protector because it is able to form a unique protecting film on the cell surface under wicked conditions such like extremely high or low temperature, high osmotic pressure and dry/water loss, thereby protecting protein molecules from denature and inactivity.

In the research and development of bioassay kits, enzyme immobilization is a critical technique. Compared with immobilized enzymes in a “solution” state, the decrease of enzyme activity of immobilized enzymes in a “dry” state is slower. So it is highly advantageous to extend the preservative time of the bioassay kit.

The coating film 120 and the stationary layer 102 use casein as the main constituting material due to its low cost. Unlike the other proteins with a stable three-dimensional structure, casein has an irregularly flexible structure, hence it has chemical properties different to other proteins; for example, it possesses properties of heat resistance and high viscosity, and it is easy to reduce its interfacial free energy. Adding calcium ions (Ca²⁺) into casein increases the interfacial adhesion of casein. Particularly, when the concentration of calcium ions reaches the threshold of 12 mM or higher, casein and calcium ions bind to each other and form a gel-like protein structure that results in an increase of the viscosity. And, when this gel-like protein structure is applied in bioassay elements, it will help enzyme immobilization and the maintenance of enzyme activity.

Particularly, the bioassay element of the present invention can be used in the pesticide residues detection based on Ellman's test. In Ellman's test, acetylcholinesterase is used to hydrolyze acetylthiocholine to thiocholine and acetic acid, then the thiocholine is reacted with the color developer DTNB (5,5′-dithio-bis-(2-nitrobenzoic acid)) to produce 5-thio-2-nitro-benzoic acid. 5-thio-2-nitro-benzoic acid has an absorption peak at 405 nm, so the activity of acetylcholinesterase can be determined by the absorbance. Since acetylcholinesterase is inhibited by pesticides such as organic phosphorus compounds and carbarmates, the color development is inhibited or reduced. Thus, Ellman's test can be used to detect pesticide residues.

The following examples are only exemplified as the best embodiments, not intended to limit the scope of the present invention. Those skilled in the art can make appropriate changes and modifications according the disclosure hereinafter without departing from the spirit of the present invention.

### Example 1

### Preparation of Bioassay Elements

A mixed solution was individually coated on porous cellulose filter papers having pore sizes of 10 μm, 23 μm and 30 μm, and the excess solution was removed by suction until these filter papers were dried and a stationary layer was formed thereon. Said mixed solution was prepared by mixing 4% of casein solution and 300 mM of a calcium ion solution in a ratio of 14:1 by volume.

1.52 U/μL of acetylcholinesterase (Sigma) solution and 5% of trehalose solution were mixed in a ratio of 2.61:108 by volume to prepare a mixed solution of enzyme. In addition, 10 mM of color developer DTNB (Sigma), 8% of casein solution and 240 mM of a calcium ion solution were mixed in a ratio of 0.009:6:1 (w/v/v) to prepare a mixed solution of casein. The mixed solution of enzyme and the mixed solution of casein were then mixed in a ratio of 5:7 by volume to prepare a bioassay solution. After that, the bioassay solution was coated on the stationary layer, and dried by means of the combination of air drying, vacuum drying and suction drying, to form a coating film in which acetylcholinesterase was embedded.

These bioassay elements of the present invention were stored at 37° C. and 4° C. for 180 days, respectively. The enzyme activity was measured every 30 days. The results show that the enzyme activity of the bioassay element stored at 37° C. and 4° C. is maintained in the range of 80%-120% after 180 days.

### Example 2

### Influence of Porous Cellulose Filter Papers Having Different Pore Sizes to Enzyme Activity

Ellman's test was performed at room temperature by using the bioassay elements produced from the three porous cellulose filter papers having different pore sizes according to Example 1. First, 6 μL of 157 mM acetylthiocholine aqueous solution, 30 μL of 10 mM DTNB solution and 964 μL of 50 mM phosphate buffer solution were added on these bioassay elements, then these element were detected at 405 nm in a spectrophotometer for 5 minutes, and the activity of the enzyme remained on the filter paper during the producing process was evaluated. The results are shown in Table 1.

TABLE 1

| | pore size (μm) | | |
|---|---|---|---|
| | 10 | 23 | 30 |
| enzyme activity | 0.58 | 0.42 | 0.36 |
| (OD₄₀₅/min.) | | | |

From the data shown in Table 1, it is known that the activity ratio of the enzyme remained on the porous cellulose filter papers having pore sizes of 10 μm, 23 μm and 30 μm is 1:0.72:0.62. As the pore size of filter paper increases, the activity of immobilized enzyme reduces. On the filter paper has a pore size of 30 μm, however, approximately 62% of the immobilized enzyme activity is remained.

### Example 3

### The Sensitivity of the Bioassay Elements Produced from Porous Cellulose Filter Papers Having Different Pore Sizes to Different Pesticides

The bioassay elements produced from the three porous cellulose filter papers having different pore sizes according to Example 1 were soaked in the solutions of three different pesticides, namely, Catbaryl, Naled and Carbofuran, for 5 minutes; wherein the concentrations of Catbaryl were 1 ppm, 3 ppm and 5 ppm, the concentrations of Naled were 0.5 ppm, 1 ppm and 3 ppm, and the concentrations of Carbofuran were 0.01 ppm, 0.05 ppm and 0.1 ppm. These pesticides were diluted in deionized water, and the controls were performed by soaking the bioassay elements in deionized water. After that, Ellman test was performed as described in Example 2, and the color changes were read by naked eyes or a spectrophotometer. The results are shown in Tables 2 to 4. In the controls, the enzyme was not inhibited by the pesticide and the bioassay element presented as dark yellow. When the enzyme in these elements was inhibited by the pesticide, the bioassay element presented as white or light yellow (data not shown).

TABLE 2
the remained enzyme activity after Carbaryl inhibition

| | |
|---|---|
| filter | concentration |

| paper pore size | control | 1 ppm | 3 ppm | 5 ppm |
|---|---|---|---|---|
| 30 μm | 0.293 | 0.070 | 0.059 | 0.048 |
| 23 μm | 0.323 | 0.087 | 0.062 | 0.061 |
| 10 μm | 0.439 | 0.24 | 0.164 | 0.092 |

TABLE 3
the remained enzyme activity after Naled inhibition

| | |
|---|---|
| filter | concentration |

| paper pore size | control | 0.5 ppm | 1 ppm | 3 ppm |
|---|---|---|---|---|
| 30 μm | 0.371 | 0.133 | 0.037 | 0.015 |
| 23 μm | 0.201 | 0.042 | 0.014 | 0.007 |
| 10 μm | 0.408 | 0.15 | 0.036 | 0.018 |

TABLE 4
the remained enzyme activity after Carbofuran inhibition

| | |
|---|---|
| filter | concentration |

| paper pore size | control | 0.01 ppm | 0.05 ppm | 1 ppm |
|---|---|---|---|---|
| 30 μm | 0.293 | 0.194 | 0.076 | 0.045 |
| 23 μm | 0.323 | 0.178 | 0.090 | 0.071 |
| 10 μm | 0.439 | 0.380 | 0.140 | 0.122 |

From the results shown in Tables 2 to 4, it is known that in all three bioassay elements differentiated by the filter paper pore size, the bioassay element produced from the filter paper having a pore size of 30 μm has the highest sensitivity, because it presented as white in all three pesticide tests. And the sensitivity next to the highest is the bioassay element using the filter paper having a pore size of 23 μm, in which Carbaryl group presents as light yellow and the other two groups present as white. As for the bioassay element using the filter paper having a pore size of 10 μm, only Carbofuran group presents as white, Naled group presents as light yellow, and the color changes in Carbaryl group is not very obvious.

From above, it is known that the smaller the filter paper pore size, the worse the pesticide inhibition sensitivity of the bioassay element. The immobilized enzyme activity of the bioassay element produced from a filter paper having a pore size of 23 μm reduced to 72%, and that of the bioassay element produced from a filter paper having a pore size of 30 μm reduced to 62%; however, both bioassay elements had excellent inhibition sensitivity to pesticides. Particularly, the bioassay element produced from a filter paper having a pore size of 30 μm is suitable for naked-eye reading.

Additionally, the detection sensibility of the bioassay element using the porous cellulose filter paper of the present invention and that of the element using nitrocellulose membrane having a pore size of 0.45 μm as part of the carrier were compared. The results are shown in Table 5. In comparison with the bioassay element comprising a nitrocellulose membrane having a pore size of 0.45 μm as part of the carrier, the bioassay element of the present invention has a lower price and higher pesticide detection sensibility.

TABLE 5
the lower limits of pesticide detection by using different substrate
materials as part of the carrier, in which the color exchanges are
read by naked eyes

| | nitrate cellulose membrane | porous cellulose filter paper |
|---|---|---|
| Carbaryl | 3 ppm | 3 ppm |
| Naled | 1 ppm | 0.5 ppm |
| Carbofuran | 0.1 ppm | 0.05 ppm |

What is claimed is:

1. A bioassay element, comprising:
a carrier comprising a porous cellulose filter paper and a stationary layer, wherein said stationary layer comprises casein and calcium ions, and covers on said porous cellulose filter paper;
a coating film comprising casein and calcium ions, which covers on the surface of said stationary layer; and
an enzyme dispersed in said coating film.

2. The bioassay element according to claim 1, wherein said porous cellulose filter paper has a pore size of 10 μm to 30 μm.

3. The bioassay element according to claim 2, wherein said porous cellulose filter paper has a pore size of 23 μm to 30 μm.

4. The bioassay element according to claim 1, wherein said enzyme is acetylcholinesterase.

5. The bioassay element according to claim 1, wherein said coating film further comprises a color developer.

6. The bioassay element according to claim 1, wherein said coating film further comprises trehalose.

7. A method of producing the bioassay element according to claim 1, which comprises steps of:
(a) providing a porous cellulose filter paper;
(b) coating a mixed solution on said porous cellulose filter paper, wherein said mixed solution comprises casein and calcium ions;
(c) drying said mixed solution to form a stationary layer;
(d) coating a bioassay solution on said stationary layer, wherein said bioassay solution comprises an enzyme, casein and calcium ions; and
(e) drying said bioassay solution.

8. The method according to claim 7, wherein said enzyme is acetylcholinesterase.

9. The method according to claim 7, wherein said mixed solution of step (c) is dried by means of air drying, vacuum drying, suction drying, or a combination thereof.

10. The method according to claim 7, wherein said mixed solution of step (b) is prepared by mixing 2.5% to 4% of casein solution and 150 mM to 300 mM of a calcium ion solution in a ratio of 14:1 to 11:1 by volume.

11. The method according to claim 7, wherein said bioassay solution of step (e) is dried by means of air drying, vacuum drying, suction drying, or a combination thereof.

12. The method according to claim 7, wherein said bioassay solution of step (d) is prepared by mixing a mixed solution of enzyme and a mixed solution of casein in a ratio of 5:7 to 5:8.75 by volume.

13. The method according to claim 12, wherein said and said mixed solution of casein comprises casein and calcium ions.

14. The method according to claim 12, wherein said mixed solution of enzyme is prepared by mixing 0.4 U/μL to 1.52 U/μL of an enzyme solution and 5% of trehalose solution in a ratio of 2:108 to 3:108 by volume.

15. The method according to claim 12, wherein said mixed solution of casein is prepared by mixing 10 mM of color developer DTNB, 4% to 10% of casein solution and 120 mM to 320 mM of a calcium ion solution in a ratio of 0.009˜0.012:6˜7.75:1 (w/v/v).
